(19) United States
(12) Patent Application Publication
(10) Pub. No.: US 2012/0184028 A1
(43) Pub. Date: Jul. 19, 2012

(54) Micro IVF chamber

(76) Inventors: John Robert Swanson, Clarence Center, NY (US); Roger A. Dugas, Chester, NH (US); Leo V. McNamara, JR., Framingham, MA (US)

(21) Appl. No.: 12/930,931

(22) Filed: Jan. 19, 2011

(51) Int. Cl. C12M 3/00

(52) U.S. Cl. 435/303.1

(57) ABSTRACT

A micro IVF chamber that enables rapid filling, hermetic sealing, and custom gas environment control for secure cell extraction, fertilization, culturing, insemination, macro and microscopic content examination, post examination rapid refilling, return to optimal gas levels and pH values, with direct view of cell in conventional microscopes while maintaining culture environment, designed for use in any incubator and multiple devices per shelf for optimum efficiency while maintaining integrity of individual devices and patient-culture identity in a simple one-sample clamp-and-go device is disclosed.

### BACKGROUND OF THE INVENTION

1. Field of the Invention

The present invention relates to a device for providing a sealed and gassed and thermally stable culturing environment in a secure compact portable chamber for In Vitro Fertilization (IVF).

2. Discussion of the Related Art

In IVF, eggs are extracted from a woman and combined in a laboratory with sperm from a man. Fertilization usually takes place in one day. The resulting embryos are kept in an incubator for three to five days, after which time they are checked for appropriate development, and then transferred into the woman's uterus.

On the day before the oocyte (egg) retrieval, oocyte culture dishes are labeled with the patients name, date of birth, and lab identification number. The number and size of the ovarian follicles determines the number of dishes prepared (one dish for each large follicle). The dishes are filled with a rinsing fluid in an outer well for removing blood and other extraneous cells from the egg, and a nutrient fluid for the culture of the egg is in a center well.

The male will be asked to collect a semen sample after the oocyte aspiration. The andrologist escorting him to the collection room will identify the patient's partner from his identification bracelet prior to collection and will write both names on the outside of the specimen container after collection. The partner will be asked to verify that both names are correct.

The embryologist will determine the number of eggs obtained and classify them as mature, immature, post mature or degenerative. The sperm is then processed in an IVF laboratory. Sperm and eggs are placed in the dishes and fertilization takes place (in vitro) outside the patient's body in the incubator.

The dishes placed in an embryology incubator are warmed to body temperature. The incubator also adjusts the pH of the culture media to the human body. The most commonly used environmental conditions for human IVF incubators are 5% CO2 in air, 37 degrees Centigrade (98.6 F), and 100% relative humidity. Regular air is about 78% nitrogen (N2) and 22% oxygen (O2). The CO2 content of air is less than one tenth of 1%. Some labs culture embryos in a low oxygen system using approximately 5% CO2, 5% O2 and 90% N2.

The fertilization process takes approximately 10-20 hours. Embryos are then cultured for 3-5 days before being replaced back in the uterus by a simple technique much like intrauterine insemination.

A variety of coverslips (a thin glass plate) and clamps are often used to cover the sample dishes to contrive individual sealed chambers within the incubator. These devices tend to be complicated, large, and cumbersome to handle and put the sample at risk of “sloshing” around inside and getting lost in the dish.

The ability to use existing incubators to store multiple chamber devices on each shelf and maintain the integrity of the culture environment and the identification of each sample as separate from each other present challenges to IVF laboratory. Incubators must maintain a very constant and clean environment for embryo culture. A large amount of rinsing media is equilibrated in the incubator. The fluid is used by the physician to wash the oocyte from the ovarian follicle if it is not found in the initial aspirations.

The simplest chamber product alternative to a cover slipped dish appears to be the Billups-Rothenberg Chamber but only one or maybe two of their chambers will fit into conventional incubators used in the target market (Human IVF). That's because the Billups-Rothenberg chamber is a multiple sample or tissue culturing system geared for the biotech laboratory that performs massive tissue culturing colonies as compared to IVF; it is large with shelves inside designed for long-term culturing and/or gas and nutrient addition during the culturing period.

Incubators are typically about the size of a small refrigerator and are often stacked to save lab space. Colored tape on the door is used to identify the location of a patients eggs or embryos in the incubator before the door is opened. This reduces the amount of time that the door has to remain open.

Typical use is to remove the culture dish from the incubator to check culture growth every 24 hours under a conventional stereo or inverted microscope and as quickly as possible review the cell's growth and return the dish to the stable culture environment of the incubator.

Current practice in IVF is the use of incubators that deliver either single gas concentrations (CO2) or a mixed-gas of O2 and CO2. Different culture media require different gas levels to maintain optimal pH. Conventional incubators struggle to achieve these gas settings after every door opening. For a variety of conventional incubators return to optimal gas levels can take from 5 to as long as 45 minutes with the resultant fluctuations in pH values. This large box recovery requires major financial investment in gas supply, storage, and delivery systems.

There is need for a compact portable hermetic sealed environmental chamber designed for the IVF lab requirement to maintain one sample per device and check culture growth every 24 hours under a conventional stereo or inverted microscope where typical use would be to remove the sealed chamber with culture dish from the incubator, remove lid, remove culture dish and examine the cell's growth. The reverse process post examination would then be performed and the lid resealed the gas inside the chamber recharged, and the chamber returned as quickly as possible to the conventional large incubator.

### SUMMARY OF THE INVENTION

It is a primary object of the present invention to overcome all the aforementioned challenges by providing a micro chamber to continuously maintain a gaseous environment of high humidity and controlled temperature around single or multiple IVF culture dishes that will support culture growth while situated, for example, within an incubator or on a microscope stage.

Another object of the invention is the provision of a thermal conductive base to hold the dish securely in place and through radiant heat absorption (device sitting in incubator or on heated plate) provide for temperature stabilization of the cell environment inside the dish.

Another object of the invention is the provision of an integrated electrical circuit thermal source to maintain a user selected temperature.

Another object of the invention is the provision of a transparent dome lid for visual inspection of the contents.

Another object of the invention is the provision of a gasket and latch mechanism to allow a pressured unique and custom gas mixture to be introduced and maintained securely inside the chamber for a minimum of 72 hours and hermetically ensure protection against ambient gas entering the chamber.

Another object of the invention is the provision of an optically clear (glass or plastic) viewing port allowing microscopic visualization of the dish contents.

Another object of the invention is the provision of a viewing port that will maintain the temperature integrity of the internal culturing environment by utilizing a heat source integrated to the glass or plastic insert that warms the dish and allows the user to set the and maintain the temperature of the glass or plastic insert.

Another object of the invention is the provision of an integrated temperature display (analog or digital) in the base to demonstrate temperature of the conductive base structure and in turn the approximate temperature of the culture environment.

Another object of the invention is the provision in the base of an integrated input and output system of fittings, connectors and valves for introducing a gas mixture to the chamber.

Another object of the invention is the provision pressure gauge connected to the output fitting to display the achieved pressure inside the chamber.

Another object of the invention is the base will be sized to fit in the palm of the hand of a typical lab technician to ensure ease of movement from storage environment (incubator, refrigerator, etc) to examining microscope or workbench.

Another object of the invention is incorporation of a bubble level mechanism into the base to display the levelness of the device.

Another object of the invention is the base will be capable of repeat sterilizations in conventional autoclaves, dishwashers, or other manual and automatic methods of cleaning and sterilization.

Another object of the invention is the dome and gasket system will be a single use item designed for a single culturing cycle and then discarded.

Another object of the invention is the base will incorporate a means to allow the user to monitor the internal temperature and gas status using independent probes integrated into the base.

Additional features of the present invention will become apparent from the following description and appended claims, taken in conjunction with the accompanying drawings.

### BRIEF DESCRIPTION OF THE DRAWINGS

Many aspects of the disclosure can be better understood with reference to the following drawings. The components in the drawings are not necessarily to scale. Moreover, in the drawings, like reference numerals designate corresponding parts throughout the several views.

FIG. 1 is a top perspective view of the chamber construction in assembled sealed relationship of this invention.

FIG. 2 is a top perspective view of the chamber base of this invention.

FIG. 3 is a bottom perspective view of the chamber base of this invention.

FIG. 4 is a top perspective view of the chamber cover of this invention.

FIG. 5 is a top plan view of the chamber base of this invention.

FIG. 6 is a bottom plan view of the chamber base of this invention.

FIG. 7 is a cross-sectional view of the chamber base construction of this invention taken through line 7-7 of FIG. 5.

FIG. 8 is a right side view of the chamber base of this invention.

FIG. 9 is a cross-sectional view of the chamber base construction of this invention taken through line 9-9 of FIG. 5.

FIG. 10 is a front view of the chamber base of this invention.

FIG. 11 is a cross-sectional view of the chamber base construction of this invention taken through line 11-11 of FIG. 6.

FIG. 12 is a cross-sectional view of the chamber base construction of this invention taken through line 12-12 of FIG. 6.

FIG. 13 is a top plan view of the chamber cover of this invention.

FIG. 14 is a cross-sectional view of the chamber cover construction of this invention taken through line 14-14 of FIG. 13.

FIG. 15 is a right side view of the chamber cover of this invention.

FIG. 16 is a front view of the chamber cover of this invention.

FIG. 17 is a side view of the control stem for the input and the output valves of this invention.

FIG. 18 is a control knob end view of the chamber input and the output valve controls of this invention.

FIG. 19 is a perspective view of the chamber base latch pivot of this invention.

FIG. 20 is a perspective view of the chamber base latch thumbscrew of this invention.

### DETAILED DESCRIPTION

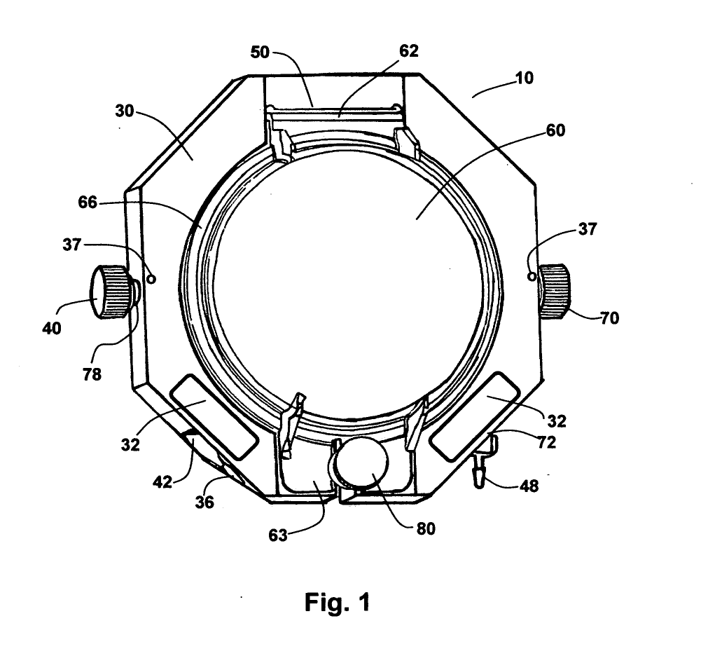

Referring to FIGS. 1-20, there is shown an illustrative embodiment of the invention device, designated generally 10, as designed for use by the embryologist to maintain an IVF culturing environment independent of ambient disruptions to ensure integrity of a compete culturing cycle.

FIG. 1 is a top perspective view of IVF culture chamber 10 which comprises octagonal shaped base 30; dome cover 60, seal o-ring 67 in cover groove 66, cover latch tab 63 retained by latch thumbscrew 80 to a pivot rotatably held in base sleeve 36, cover hinge tab 62 retained in base slot 50, output control valve 40, control valve stem 78, external output port 42, input control valve 70, external input port 72 with input hose fitting 48 threaded therein, and label recesses 32.

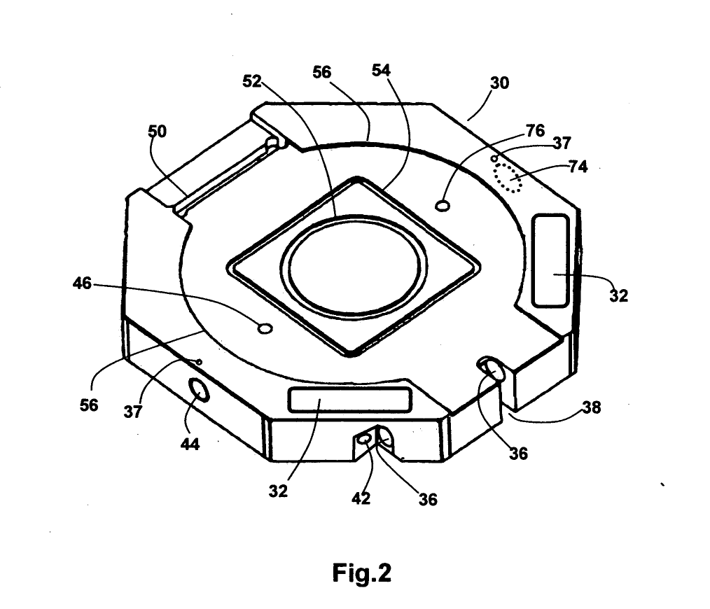

FIG. 2 is a top perspective view of uncovered octagonal shaped IVF culture chamber base 30 which comprises internal output port 46, internal input port 76, culture dish indents 52 and 54 positioned between the internal ports, major surface recess 56 surrounding the indents and internal ports, thumbscrew notch 38, latch pivot sleeve 36, cover hinge slot 50, output valve socket 44, input valve socket 74, external output port 42, thumbscrew notch 38, latch pivot sleeve 36, and label recesses 32.

FIG. 3 is a bottom perspective view of base 30 showing pedestal 31, circular grooves 33, cross grooves 35, thumbscrew pivot sleeve 36, control valve set screw 37, output control valve socket 44, thumbscrew notch 38, and cover hinge tab slot 50.

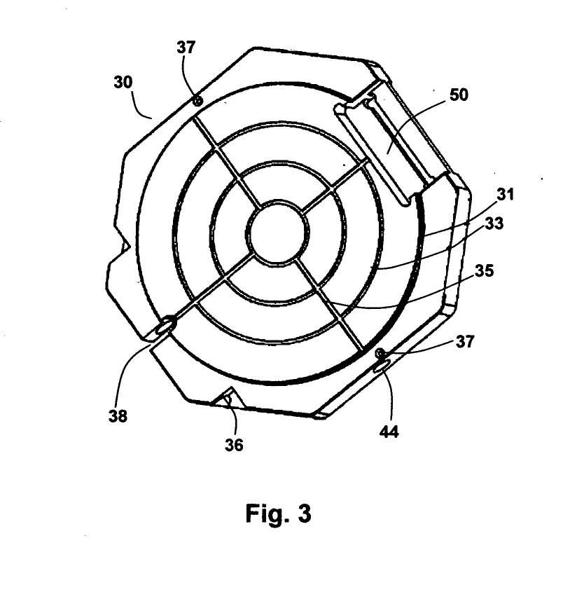

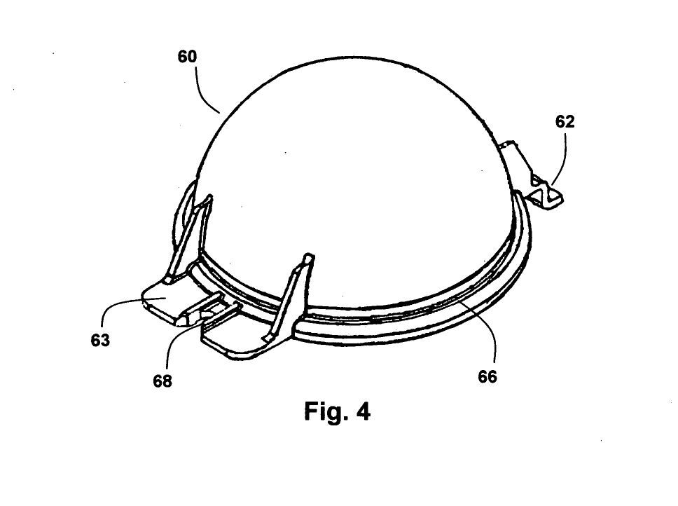

FIG. 4 is a top perspective view of dome cover 60 showing cover hinge tab 62, cover latch tab 63, thumbscrew retaining notch 68, and seal o-ring 67 in retaining groove 66.

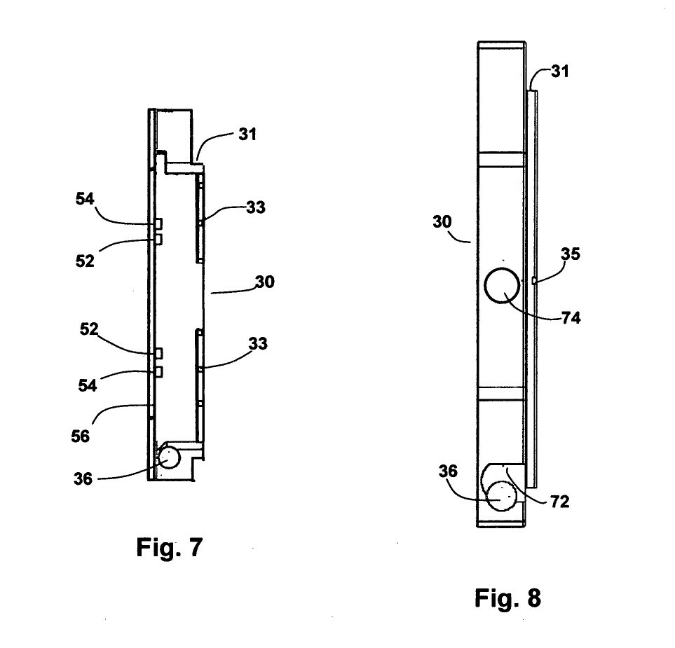

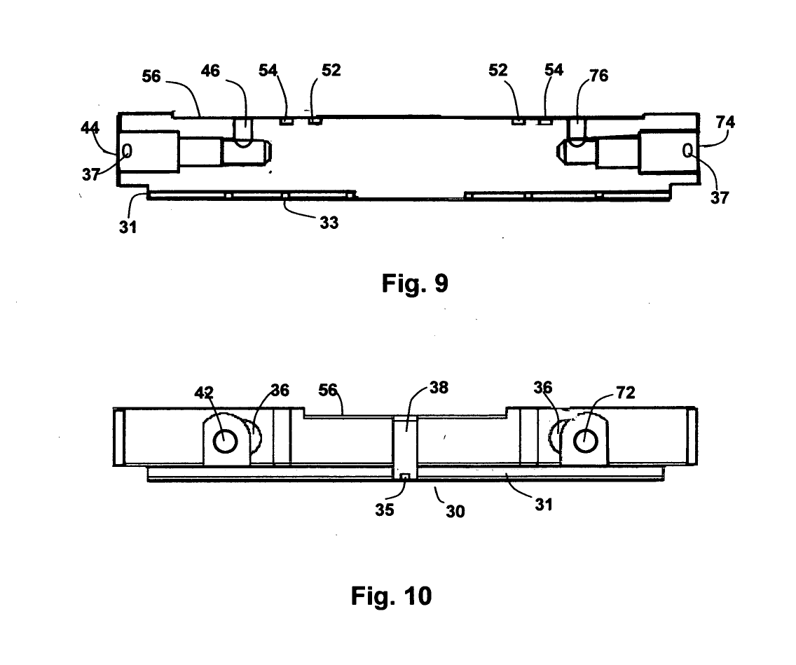

FIG. 5 is a top plan view of base 30 showing internal input port 76, internal output port 46, culture dish indents 52 and 54 placed between internal ports 76 and 46, thumbscrew notch 38, cover hinge slot 50, with line 7-7 in reference to cross-section FIG. 7 and line 9-9 in reference to cross-section FIG. 9.

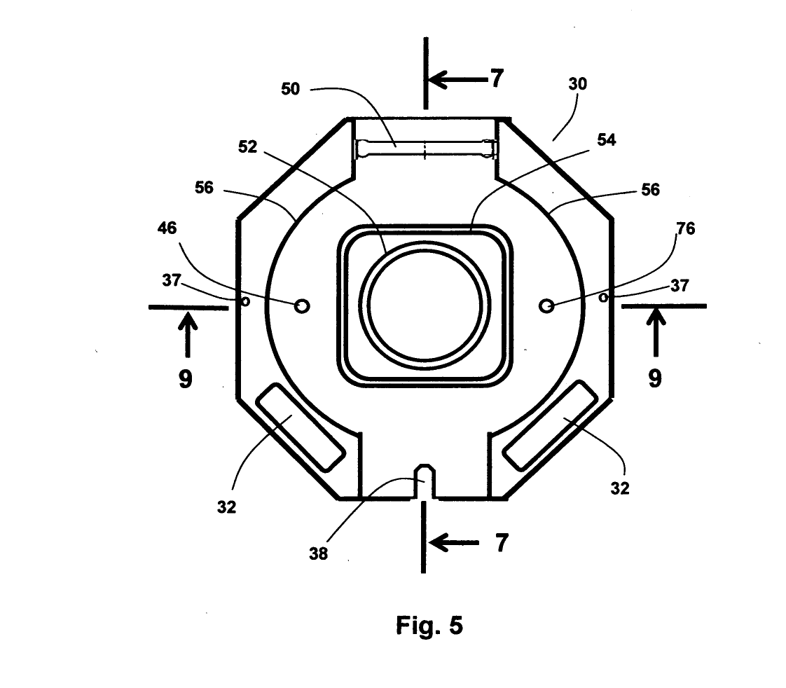

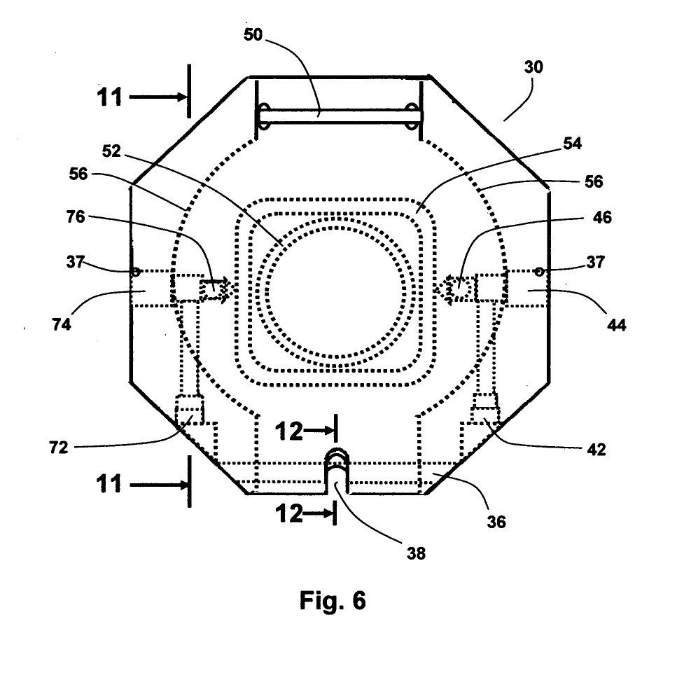

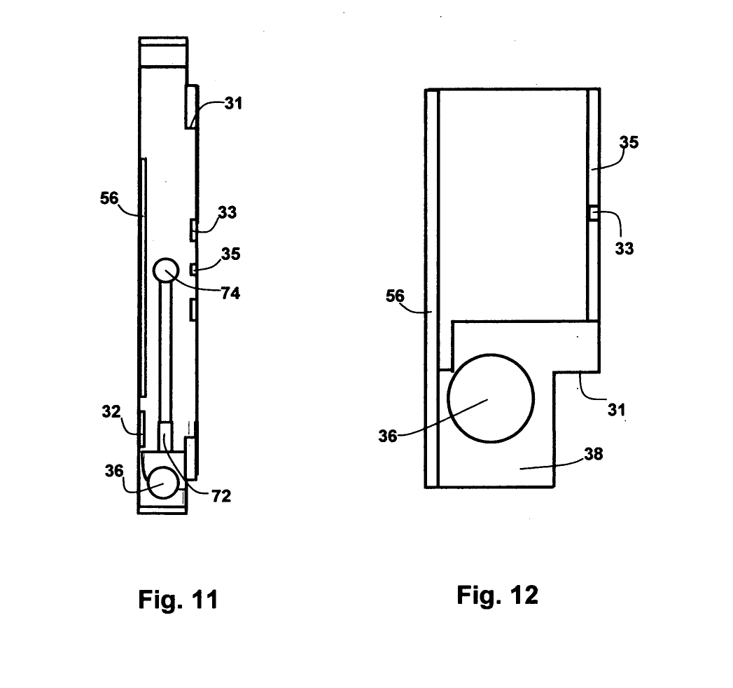

FIG. 6 is a bottom plan view of base 30 showing fluid communication between external input port 72 and internal input port 76 through control valve socket 74, fluid communication between external output port 42 and internal output port 46 through control valve socket 44, culture dish indents 52 and 54 placed between internal ports 76 and 46, thumbscrew notch 38, cover hinge slot 50, line 11-11 reference to cross-section FIG. 11, and line 12-12 reference to cross-section FIG. 12.

FIG. 7 is a cross-section view of base 30 along line 7-7 of FIG. 5 showing pedestal 31, circular grooves 33, culture dish indents 52 and 54, surrounding major surface recess 56, and latch pivot sleeve 36.

FIG. 8 is a right side view of base 30 showing pedestal 31, cross groove 35, latch pivot sleeve 36, external input port 72, and control valve socket 74.

FIG. 9 is a cross-section view of base 30 along line 9-9 of FIG. 5 showing pedestal 31, circular grooves 33, culture dish indents 52 and 54, internal port 46 with its fluid communication to control valve socket 44, internal port 76 with its fluid communication to control valve socket 74, and surrounding major surface recess 56.

FIG. 10 is a front view of base 30 showing pedestal 31, cross groove 35, latch pivot sleeve 36, thumbscrew notch 38, external output port 42, major surface recess 56, and external input port 72.

FIG. 11 is a cross-section view of base 30 along line 11-11 of FIG. 6 showing external input port 72 with its fluid communication to control valve socket 74, latch pivot sleeve 36, major surface recess 56, label recess 32, pedestal 31, and circular groove 33, cross groove 35.

FIG. 12 is a cross-section view of base 30 along line 12-12 of FIG. 6 showing pedestal 31, circular groove 33, cross groove 35, latch pivot sleeve 36, thumbscrew notch 38, and major surface recess 56.

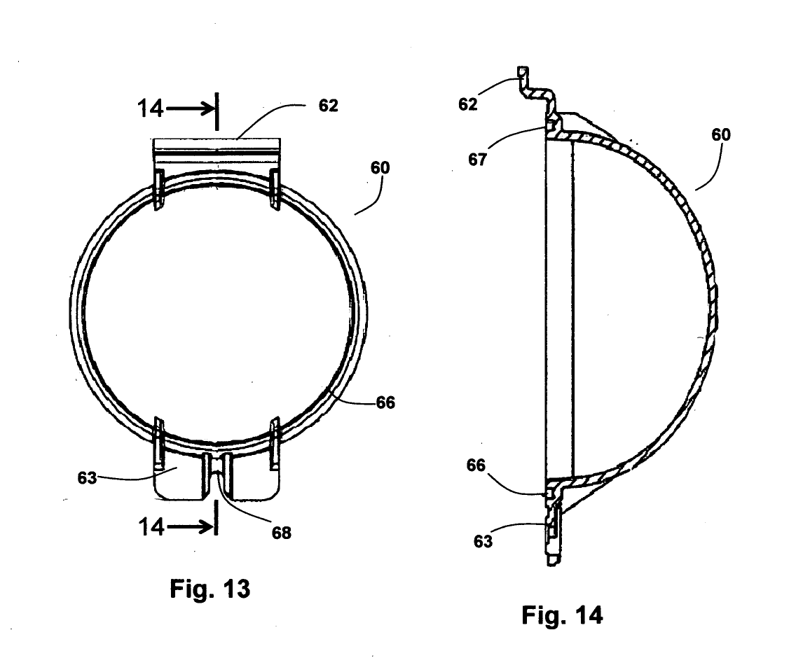

FIG. 13 is a top plan view of dome cover 60 showing cover latch tab 63, thumbscrew retaining notch 68, cover hinge tab 62, circumferential seal 67 in retaining groove 66, and line 14-14 reference to cross-section FIG. 14.

FIG. 14 is a cross-section view of dome cover 60 along line 14-14 of FIG. 13 showing cover hinge tab 62, cover latch tab 63, and seal o-ring 67 in retaining groove 66.

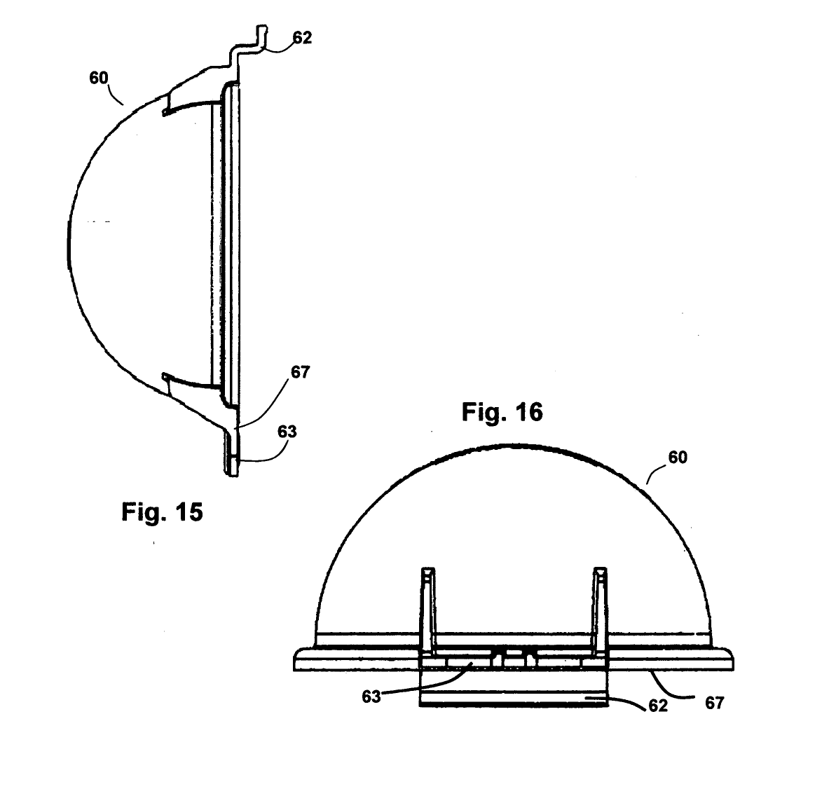

FIG. 15 is a right side view of dome cover 60 showing cover latch tab 63, cover hinge tab 62, and seal o-ring 67.

FIG. 16 is a front view of dome cover 60 showing cover latch tab 63, cover hinge tab 62, and seal o-ring 67.

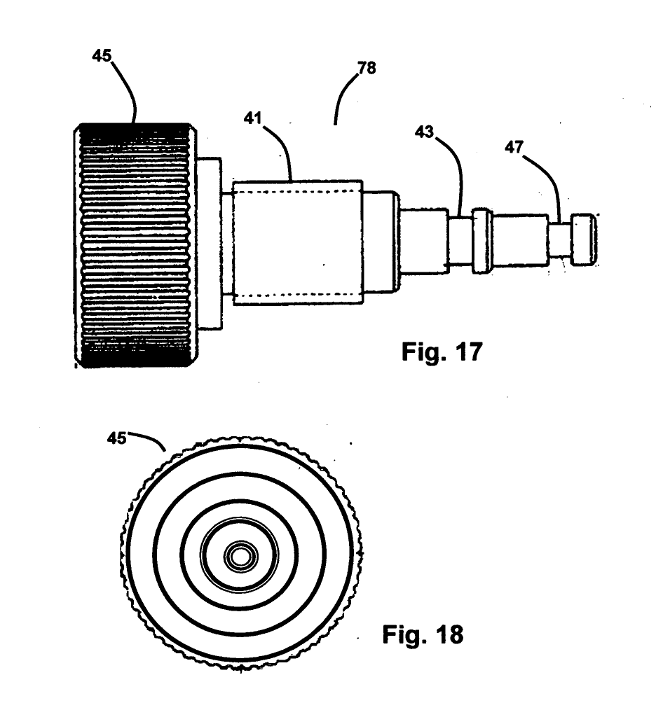

FIG. 17 is a side view of control valve stem 78 which is retained by sockets 44 and 74 for valves 40 and 70 respectively showing external port seal o-ring groove 43, internal port seal o-ring groove 47, stem thread surface 41, and valve control knurled knob 45.

FIG. 18 is a knob-end view of control valve stem 78.

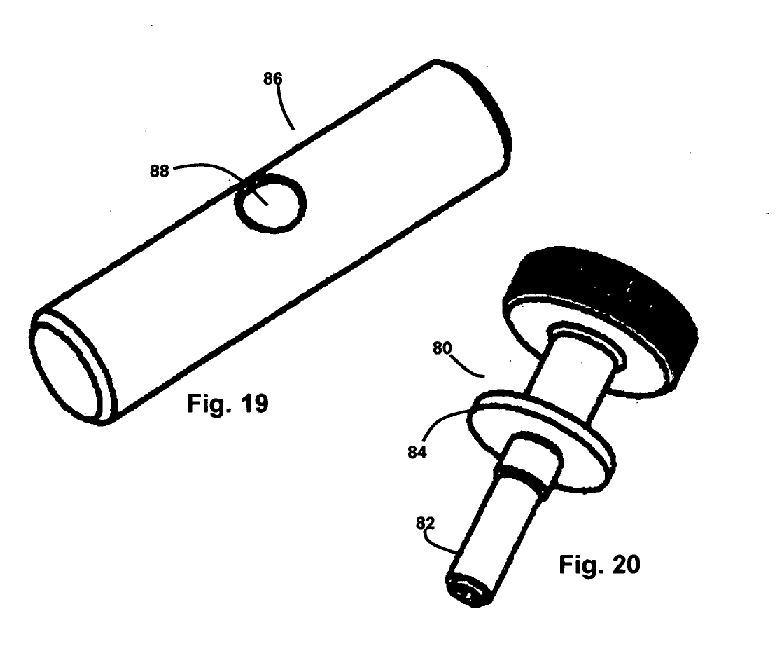

FIG. 19 is a perspective view of latch pivot 86 which is positioned rotatively in sleeve 36 of base 30 and secured longitudinally by thumbscrew 80 threaded into latch pivot hole 88 via notch 38 in base 30.

FIG. 20 is a perspective view of thumbscrew 80 having threads 82 which screw into latch pivot hole 88 to secure dome cover 60 to base 30 compressively through gasket 67 by advancing thumbscrew flange 84 against cover latch tab 63 to close hermetically chamber 10.

It is a primary object of the present invention to provide a micro chamber 10 to continuously maintain a gaseous environment of high humidity and controlled temperature around single or multiple IVF culture dishes that will support culture growth while situated, for example, within an incubator or on a microscope stage.

Micro chamber 10 is sized to fit in the palm of the hand of a typical lab technician to ensure ease of movement from an incubator to an examining microscope or workbench for use in conjunction with one or more culture dishes.

Base 30 is thermally conductive and has indents 52 and 54 to hold the culture dish securely in place to provide for temperature stabilization of the cell environment inside the dish through radiant heat absorption when the chamber 10 is sitting in an incubator or on a heated plate, the indents 52 and 54 being matched to the feet or underside of the culture dish so the underside of the dish is in full contact with base 30 thereby improving conductive temperature transfer between base 30 and the dish.

Base 30 bottom has a concentric pattern of circular and radial grooves capable of preventing thermal warp while insuring heat radiation.

Base 30 is capable of repeat sterilizations in conventional autoclaves, dishwashers, or other manual and automatic methods of cleaning and sterilization.

Domed cover 60 is made of see-through material for visual inspection of the chamber contents, cover 60 being a single use item designed for a single culturing cycle and then discarded.

Gasket 67 retained in cover groove 66 and latch mechanism tab 63 retained by thumbscrew 80 and hinge cover hinge tab 62 retained in base slot 50 allow a pressured unique and custom gas mixture to be introduced and maintained securely inside the chamber for a minimum of 72 hours and ensure protection against ambient gas entering the chamber, the gasket 67 being an O-ring of the gland type retained in cover groove 66 to provide an hermetic seal between the opposed base 30 and cover 60 which are designed as a unit for pressurization, gasket 67 being a single use item designed for a single culturing cycle and then discarded.

Gas input and output systems for purge, fill, and pressurization of sealed chamber 10 are integrated in base 30 for one-hand operation by input control valve 70 in socket 74 communicating with external input port 72 and internal input port 76 and by output control valve 40 in socket 44 communicating with internal output port 46 and external output port 42.

An error free identification system linking micro chamber 10 by text/bar code label to its culture dish is provided for by recesses 32 in base 30 outside domed cover 60.

Base top major surface recess 56 surrounds culture dish indents 52 and 54 to confine and conduct spillage to the front and rear sides of base 60 for disposal.

What is claimed is:

1. A micro chamber sized to fit in the palm of the hand of a typical lab technician to ensure ease of movement from an incubator to an examining microscope or workbench for use in conjunction with one or more culture dishes comprising:
a base thermally conductive having indents to hold the culture dish securely in place to provide for temperature stabilization of the cell environment inside the dish through radiant heat absorption when the chamber is sitting in an incubator or on a heated plate, the indents being matched to the feet or underside of the culture dish so the underside of the dish is in full contact with the base thereby improving conductive temperature transfer between the base and the dish, the base bottom having a pedestal grooved to prevent sticking while insuring heat conduction and radiation, the base top having a major recess surrounding the culture dish indents to conduct spillage to front and rear sides, the base being capable of repeat sterilizations in conventional autoclaves, dishwashers, and manual methods of cleaning and sterilization;
a domed cover made of see-through material for visual inspection of the chamber contents;
a gasket and latch mechanism to allow a pressured unique and custom gas mixture to be introduced and maintained securely inside the chamber for at least 72 hours to ensure protection against ambient gas entering the chamber, the gasket providing an hermetic seal between the opposed base and the cover which are designed as a unit for pressurization;
a gas input and output system integrated in the base comprising positive stop on/off valves, fittings, connectors, and labeling to ensure simple one-hand proper operation;
a positive identification system having outside labels recessed in the base to match text/bar code labels of the inside culture dish;

2. The domed cover of claim 1 having its material selected from the group including medical grade polycarbonate, glass, and Pyrex.

3. The base of claim 1 having an integrated analog or digital thermometer to indicate temperature of the conductive base structure and in turn the approximate temperature of the culture environment.

4. The base of claim 1 having an integrated electrical circuit thermal source and thermostat to maintain a user selected temperature.

5. The base of claim 1 having a chamber with colored liquid and an air bubble to determine if the top surface is level horizontally.

6. The base of claim 1 having integral fittings to receive probes for monitoring internal temperature and gas status.

7. The base of claim 1 having a pressure gauge to display the achieved pressure inside the chamber.

8. The base of claim 1 having a viewing port with an optically clear glass or plastic insert to allow microscopic examination of the dish contents.

9. The gasket of claim 1 being an O-ring of the gland type.

10. The gasket of claim 1 being a single use item designed for a single culturing cycle and then discarded.

11. The cover of claim 1 being a single use item designed for a single culturing cycle and then discarded

12. The viewing port according to claim 8 having a heat source and thermostat integrated with the insert that warms the dish and allows the user to set and maintain temperature of the insert in order to maintain temperature integrity of the internal culturing environment.
